(19) United States
(12) Patent Application Publication
(10) Pub. No.: US 2026/0325031 A1
(43) Pub. Date: Oct. 22, 2026

(54) PLUG-AND-PLAY BRAIN ACTIVITY INTERACTION MODULE AND APPLICATION OF BRAIN ACTIVITY INTERACTION SYSTEM THEREWITH

(71) Applicant: National Sun Yat-sen University, Kaohsiung (TW)

(72) Inventors: Yuan-Pin Lin, Kaohsiung (TW); Jan-Rung Hsu, Kaohsiung (TW); Wei-Yun Hong, Kaohsiung (TW); Yi-Hsiang Lien, Kaohsiung (TW); Shang-You Yang, Kaohsiung (TW)

(21) Appl. No.: 19/246,156

(22) Filed: Jun. 23, 2025

(30) Foreign Application Priority Data
Apr. 17, 2025 (TW) 114114573

(51) Int. Cl. A61B 5/291; A61B 5/00; A61B 5/256; A61B 5/31; A61B 5/372; A61N 1/04; A61N 1/36

(52) U.S. Cl. CPC A61B 5/291; A61B 5/256; A61B 5/31; A61B 5/372; A61B 5/6803; A61N 1/0476; A61N 1/0484; A61N 1/36025; A61N 1/3603; A61B 2560/0468; A61B 2562/046; A61B 2562/227

(57) ABSTRACT

A plug-and-play brain activity interaction module includes a microprocessor control unit, a plug-and-play first connection port electrically coupled to the microprocessor control unit, and a conversion module electrically coupled to the microprocessor control unit and communicated with an exterior brain activity terminal member. The conversion module receives a sensing signal from the exterior brain activity terminal member or outputs a control parameter to the exterior brain activity terminal member or both. The microprocessor control unit analyzes the sensing signal or generates the control parameter or both. The plug-and-play brain activity interaction module is applied to a brain activity interaction system for providing an input/output hub module to be coupled to a brain activity interaction kit for computing, analyzing and controlling.

### CROSS REFERENCES

This application claims priority to Taiwan patent application No. 114114573 filed on, April, 17, 2025, titled as “Plug-and-Play Brain Activity Interaction Module and Application of Brain Activity Interaction System therewith ”, all of which are incorporated by reference herein in its entirety.

### FIELD OF THE INVENTION

The present invention relates to a system for measuring and stimulating brain activity, particularly to a brain system with measuring and stimulating components.

### BACKGROUND OF THE INVENTION

A development of wearable brain activity sensing equipment encourages the acceptance and utilization of measuring brain signal, brain function diagnosis, brain function stimulation and brain computer interface.

Taiwan utility model patent No. M568690 discloses an assembled holder structure for positioning brain activity sensors, which provides plural sensor placement seats and detachable strips configured to assemble the sensor placement seats to implement the quantity and placement flexibilities of brain sensors for sensing brain activity.

Besides, there is still a lack of flexibility in sensor displacement and the integration of various devices for processing brain activity signal and stimulating brain function. Thus, the development of wearable brain activity sensing devices is still limited so far.

### SUMMARY OF THE INVENTION

A plug-and-play brain activity interaction module and a system therewith are provided herein, which couple each other with plug-and-play ports and transmitting cables. It is beneficial to provide flexibilities of integration and process pertaining to measurement and stimulation of brain activities.

A plug-and-play brain activity interaction module and a system therewith are provided herein, which includes a hub module with a microprocessor control unit to more flexibly integrate the measurement of brain activity and the process of brain stimulation.

Accordingly, a plug-and-play brain activity interaction module includes: a microprocessor control unit; a plug-and-play first connect port electrically coupled to the microprocessor control unit; and a converting unit electrically coupled to the microprocessor control unit and communicated with an exterior brain activity terminal component, wherein the converting unit is configured for at least one of receiving a sensing signal from the brain activity terminal component and outputting a control parameter to the brain activity terminal component, and the microprocessor control unit is configured for at least one of computing and analyzing the sensing signal and generating the control parameter.

In one embodiment, the sensing signal is inputted into the plug-and-play brain activity interaction module via the plug-and-play first connect port.

In one embodiment, the control parameter is outputted from plug-and-play brain activity interaction module via the plug-and-play first connect port.

In one embodiment, the brain activity terminal component is coupled to the plug-and-play first connect port.

In one embodiment, the plug-and-play first connect port is compliant with a communication protocol of Serial Peripheral Interface Bus (SPI).

In one embodiment, the plug-and-play brain activity interaction module is a plug-and-play brain-activity-measuring module or a brain-stimulating module.

Accordingly, a brain activity interaction system includes: an input/output hub module including plural plug-and-play first connect ports and a microprocessor control unit; and a brain activity interaction kit at least including a terminal component and an intermediate module, wherein the terminal component is electrically coupled to the intermediate module, the intermediate module includes a plug-and-play second connect port and a pluggable first transmitting cable, the first transmitting cable is pluggable fixed to the plug-and-play second connect port and one of the plug-and-play first connect ports, and the terminal component is electrically coupled to the microprocessor control unit via the intermediate module and the plug-and-play first connect port which the first transmitting cable is fixed to.

In one embodiment, the plug-and-play first connect ports and the plug-and-play second connect port are compliant with a communication protocol of Serial Peripheral Interface Bus (SPI).

In one embodiment, the brain activity interaction kit further includes a brain headset, and the brain headset includes plural displacement seats and plural detachable strips coupled with the displacement seats, and the terminal component is deposited on one of the displacement seats.

In one embodiment, the input/output hub module and the intermediate module are individual.

In one embodiment, the terminal component is a sensor component, a stimulus component, or both.

In one embodiment, the intermediate module is a brain-activity-measuring module and the terminal component is a sensing electrode element for sensing brainwaves, and wherein according to a control comment from the microprocessor control unit, the brain-activity-measuring module controls the sensing electrode element to make a measurement of brainwave or contact impedance of scalp.

In one embodiment, the intermediate module is a brain-stimulating module and the terminal component is a stimulating electrode element, and wherein according to a control comment from the microprocessor control unit, the brain-stimulating module controls the stimulating electrode element to deliver electrical stimulus or make a measurement of contact impedance of scalp.

Accordingly, a brain activity interaction system includes: an input/output hub module including plural plug-and-play first connect ports and a microprocessor control unit; a brain-activity-measuring module including a plug-and-play second connect port and a pluggable first transmitting cable, the first transmitting cable is pluggable fixed to the plug-and-play second connect port and one of the plug-and-play first connect ports; a brain-stimulating module including a plug-and-play third connect port and a pluggable second transmitting cable, the second transmitting cable is pluggable fixed to the plug-and-play third connect port and another one of the plug-and-play first connect ports; and plural terminal components electrically coupled to the microprocessor control unit via the brain-activity-measuring module and the brain-stimulating module.

In one embodiment, the plug-and-play first connect ports, the plug-and-play second connect port and the plug-and-play third connect port are compliant with a communication protocol of Serial Peripheral Interface Bus (SPI).

In one embodiment, a brain headset is further included to include plural displacement seats and plural detachable strips coupled with the displacement seats, and the terminal components are deposited on the displacement seats.

In one embodiment, the input/output hub module, the brain-activity-measuring module and the brain-stimulating module are individual.

In one embodiment, the terminal components include plural sensor components and plural stimulus components.

In one embodiment, a portion of the terminal components are one or more sensing electrode elements for sensing brainwaves, and wherein according to a control comment from the microprocessor control unit, the brain-activity-measuring module controls the one or more sensing electrode elements to make a measurement of brainwaves or contact impedance of scalp.

In one embodiment, a portion of the terminal components are one or more stimulating electrode elements, and wherein according to a control comment from the microprocessor control unit, the brain-stimulating module controls the one or more stimulating electrode elements to deliver electrical stimulus or make a measurement of contact impedance of scalp.

### BRIEF DESCRIPTION OF THE DRAWINGS

FIG. 1 is a schematical diagram illustrating a brain activity interaction system in accordance with the present invention.

FIG. 2 is a schematical block diagram illustrating the first exemplary signal transmission and parts amid the brain activity interaction system in accordance with the present invention.

FIG. 3 is a schematically block diagram illustrating the second exemplary signal transmission and parts amid the brain activity interaction system in accordance with the present invention.

### DETAILED DESCRIPTION OF THE PREFERRED EMBODIMENT

In the present invention, so-called hot swappable or plug-and-play connect port is a connect port safely physically connected or detached without power off. In the present invention, these plug-and-play connect ports are deposited on respective separately or individual modules, and these individual modules are physically coupled with the plug-and-play connect ports. Next, there are transmitting cables coupled with the individual modules and mounted on the plug-and-play connect ports. Both of the transmitting cables and the plug-and-play connect ports are compliant with a communication protocol of serial communication, for example but not limited to, Serial Parallel Interface (SPI), I²C bus, and so on, which provide a two-way communication interface.

In the present invention, so-called brain activity measuring device performs tasks in a way of non-invasive measurement, for example but not limited to, Electroencephalography (EEG), Magnetoencephalography (MEG), Functional Near Infrared Spectroscopy (fNIRS), and Functional Magnetic Resonance Imaging (fMRI).

In the present invention, so-called brain activity stimulating device performs tasks in a way of non-invasive stimulation, for example but not limited to, Transcranial Direct Current Stimulation (tDCS), Transcranial Alternating Current Stimulation (tACS), Transcranial Magnetic Stimulation (tMS), acousto-optical simulation, and so on.

In the present invention, so-called terminal component is the one which is closest to or reaches to a brain zone for the brain activity measuring or stimulating devices and is a sensing component or a stimulating component or both. A sensor may sense brain activity in a light, electric, or magnetic way. Following sensing components may be used to sense brainwaves and measure contact impedance, such as noninvasively wet electrodes or noninvasively dry electrodes. So-called stimulator in the present invention may stimulate a brain in a light, electric, magnetic or acoustic way. Following stimulating components may be used to output direct current stimulus or along with the measurement of contact impedance, such as noninvasively sponge electrodes, noninvasively gel electrodes, or noninvasively dry electrodes. So-called intermediate module is to couple the terminal components and a hub module with a microprocessor control unit, such as a convert module for processing the measurement of brain activity or brain stimulation, which may be separated from the hub module or integrated into the hub module and include plug-and-play connect ports. The hub module in the present invention includes the microprocessor control unit and plural plug-and-play connect ports. Accordingly, in the case of the hub module with many plug-and-play connect ports, the quantity of the intermediate modules coupled to the hub module is flexibly adjusted. For example, these intermediate modules are detachably mounted onto these plug-and-play connect ports via the transmitting cables of four-role wires. Next, data communication or signal transmission between the terminal components and the intermediate modules are wired or wireless. In some embodiment, in the case of the intermediate modules integrated into the hub module, the terminal components also perform wired or wireless data communication or signal transmission.

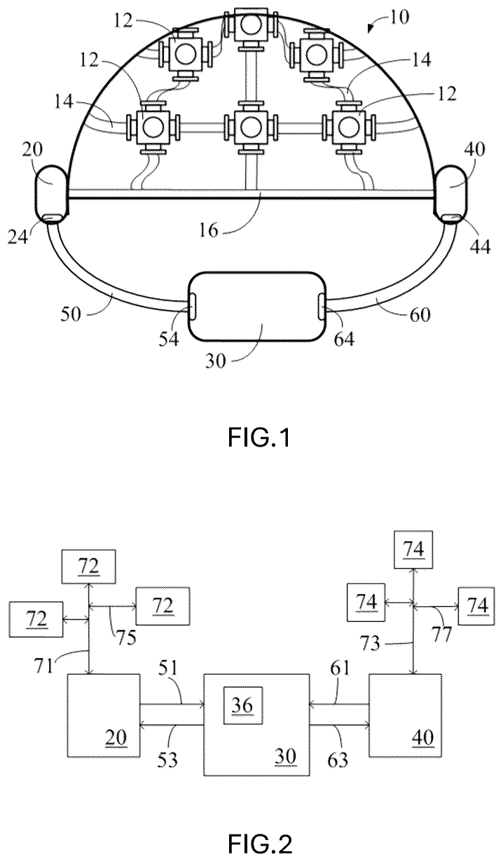

FIG. 1 is a schematical diagram illustrating a brain activity interaction system in accordance with the present invention. FIG. 2 is a schematical block diagram illustrating the first exemplary signal transmission and parts amid the brain activity interaction system in accordance with the present invention. Please refer to FIG. 1. An exemplary brain activity interaction system at least includes a brain headset 10, a brain-activity-measuring module 20, an input/output hub module 30 (called as I/O hub module 30 hereafter) and a brain-stimulating module 40. The brain headset 10 respectively couples to the brain-activity-measuring module 20 and the brain-stimulating module 40 via one or more main connect belts 16. The brain-activity-measuring module 20, such as an Electroencephalogram (EEG) module, couples to the I/O hub module 30 via a first transmitting cable 50. The brain-stimulating module 40, such as a Transcranial Direct Current Stimulation(tDCS) module, couples to the I/O hub module 30 via a second transmitting cable 60. In one embodiment, the I/O hub module 30 includes plural plug-and-play first connect port 54 and plug-and-play first connect port 64. The brain-activity-measuring module 20 and the brain-stimulating module 40 respectively include a plug-and-play second connect port 24 and a plug-and-play third connect port 44. Accordingly, the first transmitting cable 50 is detachable coupled to the first connect port 54 and the second connect port 24. The second transmitting cable 60 is detachable and coupled to the first connect port 64 and the third connect port 44. It is understood that in the example of the I/O hub module 30 equipped with the pluralities of first connect ports 54 and 64, the I/O hub module 30 may couple to the pluralities of brain-activity-measuring modules 20 or brain-stimulating modules 40.

Continuously refer to FIG. 1. The brain headset 10 at least includes plural displacement seats 12, detachable strips 14 and at least one main connect belt 16. Any one of the detachable strips 14 couples between any one of the displacement seats 12 and the main connect belt 16, or between two adjacent displacement seats 12, or between any two of the displacement seats 12. Accordingly, the brain headset 10 consists of the displacement seats 12, and any one of the displacement seats 12 may deposit one or more components such as sensor, stimulator or other functional component. Any one of the detachable strips 14 and the main connect belt 16 may have respectively embedded wires or load wires thereon or be without wires. Terminal components deposited in the displacement seats 12 are electrically coupled to the embedded or loading wires, so as to further electrically coupled to at least one of the brain-activity-measuring module 20 and the brain-stimulating module 40. The brain headset 10 may include different quantities of the displacement seats 12, so the number of the brain-activity-measuring module 20 and the brain-stimulating module 40 are adjusted depending on requirement. The terminal components in the displacement seats 12 may also wired or wirelessly communicate with the brain-activity-measuring module 20 or the brain-stimulating module 40.

Continuously refer to FIG. 1 and FIG. 2. Many sensors 72, such as brainwave sensors, are respectively deposited in any one of the displacement seats 12, and many stimulators 74, such as electrode component, are also respectively deposited in any one of the displacement seats 12. There are one sensor 72 and one stimulator 74 simultaneously deposited in same displacement seat 12, or one sensor 72 or one stimulator 74 sole deposited in one displacement seat 12. Next, the first sensing signal 71 and the first outputting signal 75 are transmitted between any one of the sensors 72 and the brain-activity-measuring module 20 via assembling detachable strips 14 and wires of the main connect belt 16(not shown in the drawing). The second sensing signal 73 and the second outputting signal 77 are transmitted between any one of the stimulators 74 and the brain-stimulating module 40 via assembling detachable strips 14 and wires of the main connect belt 16(not shown in the drawing). On the other side, the first inputting signal 51 and the first controlling signal 53 are transmitted between the brain-activity-measuring module 20 and the I/O hub module 30 via the detachable first transmitting cable 50. The second inputting signal 61 and the second controlling signal 63 are transmitted between the brain-stimulating module 40 and the I/O hub module 30 via the detachable second transmitting cable 60. It is understood, the numbers of the detachable first transmitting cable 50 and the detachable second transmitting cable 60 are variedly adjusted in the wake of the brain-activity-measuring module 20, the I/O hub module 30 and the brain-stimulating module 40 respectively equipped with the plug-and-play second connect port 24, the first connect ports 54 and 64, and the third connect port 44. For example, the I/O hub module 30 couples with only one or more brain-activity-measuring modules 20, or only one or more brain-stimulating modules 40, or simultaneously the brain-activity-measuring module 20 and the brain-stimulating module 40. In practice, the I/O hub module 30 couples with and further integrates a brain activity measurement kit and a brain-stimulating kit via the design of plug-and-play connectors for connection flexibility. The brain activity measurement kit, such as EEG kit, includes plural sensors 72 and corresponding wires, the brain-activity-measuring module 20 and the first transmitting cable 50. The sensors 72 and the corresponding wires may be along with the corresponding displacement seats 12, detachable strips 14 and the main connect belt 16. The brain-stimulating kit, such as tDCS stimulating kit, includes plural stimulators 74 and the corresponding wires, the brain-stimulating module 40 and the second transmitting cable 60. The stimulators 74 and the corresponding wires may be along with the corresponding displacement seats 12, detachable strips 14 and the main connect belt 16.

Continuously refer to FIG. 1 and FIG. 2. In one embodiment, from outwards appearance, the brain-activity-measuring module 20, the I/O hub module 30 and the brain-stimulating module 40 are individual and separated structures: respective cases, respective printed circuit boards with circuit and components in the respective cases, and detachable connecting ports on the respective cases. For signal processing, the brain-activity-measuring module 20 receives the first sensing signal 71 from the sensors 72 and outputs the first inputting signal 51 to the I/O hub module 30. The brain-activity-measuring module 20 also receives the first controlling signal 53 from the I/O hub module 30 and outputs the first outputting signal 75 to the sensors 72. In one embodiment, the first sensing signal 71, such as EEG analog signal, is analog, and the first inputting signal 51 is digital. The brain-activity-measuring module 20 includes converting module, components and circuits for processing the first sensing signal 71, for example but not limited to, signal amplification, filtering and analog/digital conversion or waveform feature calculation, and so on. Next, the I/O hub module 30 at least includes a microprocessor control unit 36 (called as MCU 36 hereafter) and corresponding components and circuits, which receives the first inputting signal 51 from the brain-activity-measuring module 20 and respectively outputs the first controlling signal 53 and the second controlling signal 63 to the brain-activity-measuring module 20 and the brain-stimulating module 40. In one embodiment, the MCU 36 of the I/O hub module 30 may process at least two switch modes for the brain-activity-measuring module 20, one kind of mode is the measurement of brain activity such as EEG measurement, and the other kind of mode is the measurement of contact impedance for brainwave electrodes on a scalp. In the case of the measurement of contact impedance by the MCU 36 of the I/O hub module 30, the first controlling signal 53 as a commend of impedance measurement outputs to the brain-activity-measuring module 20. In response to the commend of impedance measurement, the brain-activity-measuring module 20 outputs the first outputting signal 75 as a testing signal, such as low current to the sensors 72 for processing impedance measurement. It is understood that in the mode of impedance measurement, the first sensing signal 71 outputted from the sensors 72 are impedance values, and the impedance values, like brainwave analog signal, is processed by the brain-activity-measuring module 20 and transmitted to the I/O hub module 30 via the first transmitting cable 50.

Continuously refer to FIG. 1 and FIG. 2. For the brain-stimulating module 40, the MCU 36 of the I/O hub module 30 at least provides electrical parameters necessary for processing the brain stimulation, for example but not limited to, current intensity, duration, and so on, and the electrical parameters in the form of the second controlling signal 63 are transmitted to the brain-stimulating module 40 via the second transmitting cable 60. The brain-stimulating module 40 outputs corresponding direct current according to the control commends of the second controlling signal 63, and the corresponding direct current in the form of the second output signal 77 to the connected stimulators 74. In the case of the stimulators 74 of measurement function, such as tDCS electrodes of the function of contact impedance measurement, the stimulators 74 measures contact impedance and transmits the contact impedance in the form of the second sensing signal 73 to the brain-stimulating module 40, and the contact impedance is converted to be in the form of the second inputting signal 61 transmitted to the I/O hub module 30. Accordingly, the brain-stimulating module 40 may include, for example but not limited to, converting components and circuits relative of digital impedance modulation, current output processing.

Continuously refer to FIG. 1 and FIG. 2. Considering integrating effects, the MCU 36 of the I/O hub module 30, which has an edge computing framework, may analyze the digital brainwaves from the brain-activity-measuring module 20, determines electrical parameters necessary for controlling brain stimulation according to an analysis result of the digital brainwaves, and transmits control commends relative of the electrical parameters to the brain-stimulating module 40. The brain-stimulating module 40 outputs corresponding direct current according to the control commends to the stimulator 74. Alternatively, the I/O hub module 30 also executes the detection of contact impedance via sensor and stimulators. The MCU 36 respectively transmits the control commends in the form of the first controlling signal 53 and the second controlling signal 63 to the brain-activity-measuring module 20 and the brain-stimulating module 40, and then the control commends respectively reach to the sensors 72 and the simulators 74 for detection of contact impedance. The detection of contact impedance may be used to check the compliance of the electrode deposition and ensure the measurement quality of the brain activity and the delivery efficiency of the brain stimulation. Accordingly, the I/O hub module 30 with the MCU 36 provides the measurement of the brain activity and the brain stimulation with operations of an open or a close architecture, and is of scalability by providing detachable connect ports.

Continuously refer to FIG. 1 and FIG. 2. In practice, the MCU 36 of the present invention may be implemented by commercially available Raspberry Pi Zero 2, which includes microprocessor, memory, communication and storing units. In addition to the MCU 36 and the first connect ports 54 and 64, the I/O hub module 30 may include other separated modules, for example but not limited to, power supply or battery module. Next, in order to be compliant with some serial communication protocols such as SPI, there are some necessary conversion circuits, elements and modules, such as circuits and electrical elements for data/information conversion process, in the brain-activity-measuring module 20, the I/O hub module 30 and the brain-stimulating module 40, for example, microprocessor or ASIC, and so on. Next the positions of the displacement seats 12 of the brain headset 10 may be set according to the EEG electrode positions disclosed in International 10-20 Electrode System. Considering the differences to human brain, the distances amid the displacement seats 12 may be adjusted with the detachable strips 14. Besides, some displacement seats 12 may be vacant without depositing any components in the brain headset 10. Accordingly, the brain activity interaction system of the present invention adopts plug-and-play framework in which all modules and circuits are mixing built. For example, the MCU 36 may be implemented with low level MCU only for the measurement of brainwaves, or replaced by high level MCU for the process of brain-wave edge computing for integrating application of brain-computer interface.

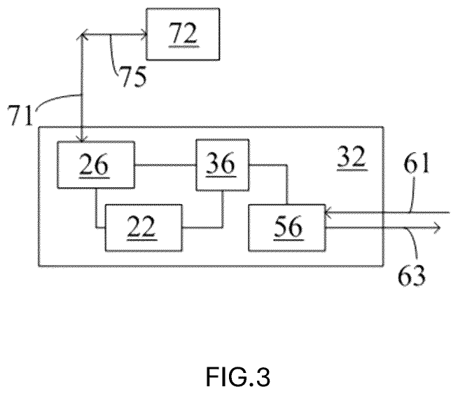

FIG. 3 is a schematically block diagram illustrating the second exemplary signal transmission and parts amid the brain activity interaction system in accordance with the present invention. Compared to FIG. 2, a brain activity measurement and input/output module 32 in FIG. 3 integrates the brain-activity-measuring module 20 and the input/output module 30 in FIG. 2, which includes a microprocessor control unit 36, a converting unit 22, a brain activity communicating module 26 and one or more first connect ports 56. The microprocessor control unit 36 electrically couples with the converting unit 22, the brain activity communicating module 26 and the first connect ports 56, respectively. In a second embodiment, one or more sensors 72 wired or wirelessly transmit one or more first sensing signals 71 to the brain activity communicating module 26 of the brain activity measurement and input/output module 32. After processed with exemplary communication protocol in the brain activity communicating module 26, the first sensing signals 71 is outputted to the converting unit 22 for conversion process, such as signal amplification, filtering and analog/digital conversion or waveform feature calculation, and so on, and then outputted to the microprocessor control unit 36 for numerical operation and analysis. Next, the microprocessor control unit 36 is used to output data after numerical operation and analysis to the outside of the brain activity measurement and input/output module 32, generate and transmit control parameter comments as the second control signal 63 to the first connect port 56, or generate and transmit control parameter comments to the brain activity communicating module 26. It is understood that in the case of other brain activity or brain stimulating intermediate module (not shown in the drawing) pluggable and coupled to the first connect ports 56, a second inputting signal 61 from the other brain activity or brain stimulating intermediate module may be transmitted to the microprocessor control unit 36 via the first connect ports 56 for numerical operation and analysis. It is also understood that in another embodiment, the input/output hub module 30 and the brain-stimulating module 40 in FIG. 2 may be integrated together to acquire one similar to the brain activity measurement and input/output module 32 in FIG. 3, which is not repeatedly illustration herein. It is also understood that some modules in the brain activity measurement and input/output module 32 may be combined, such as the brain activity communicating module 26 integrated with the converting unit 22, or the brain activity communicating module 26 integrated with the first connect ports 56, or the converting unit 22 integrated with the microprocessor control unit 36, and so on.

Accordingly, the plug-and-play brain activity interaction module of the present invention includes a microprocessor control unit, a plug-and-play first connect port and a converting unit. The plug-and-play first connect port is electrically coupled to the microprocessor control unit. The converting unit is electrically coupled to the microprocessor control unit and communicated with an exterior brain activity terminal component, which is configured for receiving a sensing signal from the brain activity terminal component, outputting a control parameter to the brain activity terminal component or both of the functions aforementioned. The microprocessor control unit computes and analyzes the sensing signal, or generates the control parameter, or executes the tasks aforementioned.

The above description is merely exemplary of the preferred embodiments of the present invention and should not be construed as limiting the scope of the invention. Any modifications, equivalents, substitutions, or improvements made within the spirit and principles of the present invention should be encompassed within the scope of protection of the invention.

What is claimed is:

1. A plug-and-play brain activity interaction module, comprising:
a microprocessor control unit;
a plug-and-play first connect port electrically coupled to the microprocessor control unit; and
a converting unit electrically coupled to the microprocessor control unit and communicated with an exterior brain activity terminal component, wherein the converting unit is configured for at least one of receiving a sensing signal from the brain activity terminal component and outputting a control parameter to the brain activity terminal component, and the microprocessor control unit is configured for at least one of computing and analyzing the sensing signal and generating the control parameter.

2. The plug-and-play brain activity interaction module according to claim 1, wherein the sensing signal is inputted into the plug-and-play brain activity interaction module via the plug-and-play first connect port.

3. The plug-and-play brain activity interaction module according to claim 1, wherein the control parameter is outputted from plug-and-play brain activity interaction module via the plug-and-play first connect port.

4. The plug-and-play brain activity interaction module according to claim 1, wherein the brain activity terminal component is coupled to the plug-and-play first connect port.

5. The plug-and-play brain activity interaction module according to claim 1, wherein the plug-and-play first connect port is compliant with a communication protocol of Serial Peripheral Interface Bus (SPI).

6. The plug-and-play brain activity interaction module according to claim 1, wherein the plug-and-play brain activity interaction module is a plug-and-play brain-activity-measuring module or a brain-stimulating module.

7. A brain activity interaction system, comprising:
an input/output hub module including plural plug-and-play first connect ports and a microprocessor control unit; and
a brain activity interaction kit at least including a terminal component and an intermediate module, wherein the terminal component is electrically coupled to the intermediate module, the intermediate module comprises a plug-and-play second connect port and a pluggable first transmitting cable, the first transmitting cable is pluggable and fixed to the plug-and-play second connect port and one of the plug-and-play first connect ports, and the terminal component is electrically coupled to the microprocessor control unit via the intermediate module and the plug-and-play first connect port which the first transmitting cable is fixed to.

8. The brain activity interaction system according to claim 7, wherein the plug-and-play first connect ports and the plug-and-play second connect port are compliant with a communication protocol of Serial Peripheral Interface Bus (SPI).

9. The brain activity interaction system according to claim 7, wherein the brain activity interaction kit further comprises a brain headset, the brain headset comprises plural displacement seats and plural detachable strips coupled with the displacement seats, and the terminal component is deposited on one of the displacement seats.

10. The brain activity interaction system according to claim 7, wherein the input/output hub module and the intermediate module are individual.

11. The brain activity interaction system according to claim 7, wherein the terminal component is a sensor component, a stimulus component, or both.

12. The brain activity interaction system according to claim 7, wherein the intermediate module is a brain-activity-measuring module and the terminal component is a sensing electrode element for sensing brainwaves, and wherein according to a control comment from the microprocessor control unit, the brain-activity-measuring module controls the sensing electrode element to make a measurement of brainwaves or contact impedance of scalp.

13. The brain activity interaction system according to claim 7, wherein the intermediate module is a brain-stimulating module and the terminal component is a stimulating electrode element, and wherein according to a control comment from the microprocessor control unit, the brain-stimulating module controls the stimulating electrode element to deliver electrical stimulus or make a measurement of contact impedance of scalp.

14. A brain activity interaction system, comprising:
an input/output hub module including plural plug-and-play first connect ports and a microprocessor control unit;
a brain-activity-measuring module comprising a plug-and-play second connect port and a pluggable first transmitting cable, the first transmitting cable is pluggable and fixed to the plug-and-play second connect port and one of the plug-and-play first connect ports;
a brain-stimulating module comprising a plug-and-play third connect port and a pluggable second transmitting cable, the second transmitting cable is pluggable and fixed to the plug-and-play third connect port and another one of the plug-and-play first connect ports; and
plural terminal components electrically coupled to the microprocessor control unit via the brain-activity-measuring module and the brain-stimulating module.

15. The brain activity interaction system according to claim 14, wherein the plug-and-play first connect ports, the plug-and-play second connect port and the plug-and-play third connect port are compliant with a communication protocol of Serial Peripheral Interface Bus (SPI).

16. The brain activity interaction system according to claim 14 further comprising a brain headset, wherein the brain headset comprises plural displacement seats and plural detachable strips coupled with the displacement seats, and the terminal components are deposited on the displacement seats.

17. The brain activity interaction system according to claim 14, wherein the input/output hub module, the brain-activity-measuring module and the brain-stimulating module are individual.

18. The brain activity interaction system according to claim 14, wherein the terminal components comprise plural sensor components and plural stimulus components.

19. The brain activity interaction system according to claim 14, wherein a portion of the terminal components are one or more sensing electrode elements for sensing brainwaves, and wherein according to a control comment from the microprocessor control unit, the brain-activity-measuring module controls the one or more sensing electrode elements to make a measurement of brainwaves or contact impedance of scalp.

20. The brain activity interaction system according to claim 14, wherein a portion of the terminal components are one or more stimulating electrode elements, and wherein according to a control comment from the microprocessor control unit, the brain-stimulating module controls the one or more stimulating electrode elements to deliver electrical stimulus or make a measurement of contact impedance of scalp.
Plug-and-Play Brain Activity Interaction Module and Application of Brain Activity Interaction System therewith
